(19) United States
(12) Patent Application Publication
(10) Pub. No.: US 2011/0189747 A1
(43) Pub. Date: Aug. 4, 2011

(54) PROCESS FOR PRODUCING SILAGE FOR BIOGAS PRODUCTION

(75) Inventors: David Seale, Caversham (GB); Inge Knap, Broenshoej (DK)

(73) Assignee: CHR-HANSEN A/S

(21) Appl. No.: 13/056,846

(22) PCT Filed: Jul. 31, 2009
(86) PCT No.: PCT/EP2009/059956

§ 371 Date: Apr. 25, 2011

(30) Foreign Application Priority Data
Aug. 5, 2008 (EP) 08104965.2

(51) Int. Cl. C12P 1/04

(52) U.S. Cl. 435/170

(57) ABSTRACT

The invention provides the use of a lactic acid bacterium and optionally a Bacillus for producing a silage which may be advantageously used for the production of biogas. The invention also provides the use of a lactic acid bacterium and optionally a Bacillus for producing biogas.

### FIELD OF THE INVENTION

The invention relates to the field of renewable energy production, specifically, biogas production. The invention provides a process for producing a silage which can be used in a process for producing biogas using a lactic acid bacterium and optionally a Bacillus. The invention also provides a process for producing biogas using a lactic acid bacterium and optionally a Bacillus.

### BACKGROUND OF THE INVENTION

In recent years, biogas has emerged as an important renewable energy source. Biogas, which primarily comprises methane (CH₄), is produced from anaerobic digestion or fermentation of biodegradable materials or biomass for electricity generation and/or for feeding to the gas grid.

To produce biogas, biomass, which serves as the substrate, is mixed with a starter or inoculum in an anaerobic digester to allow fermentation of the biomass. Most (95%) of the biogas is collected from the anaerobic digester and a small fraction (5%) from the storage tank. The remaining undigested materials are used as fertilizer (FIG. 1).

The process of biogas production is a continuous process. Biomass typically takes 7 weeks to go through the digester. The digester has to be “fed” every day with new biomass. Cattle manure is commonly used as the starter. Most of the biogas is generated in the first 3 weeks of the fermentation process.

Biomass useful for biogas production includes agricultural materials, such as liquid manure, dung, fresh plants or plant parts (e.g., grass, clover, maize, straw, sugar beet and potato leaves), silages (e.g., grass, maize), industrial residues (e.g., stillage, pomace, whey, vegetable residues, grease trap contents), and municipal waste.

It has been shown that different plants have different efficiencies in biogas production (Hermann C et al., 2007). Furthermore, parameters such as harvest time and ensiling process affect the biogas yield (Hermann C. et al., 2007).

80-90% of biogas plants use silage as substrate, but very few use silage on its own, at least at the initial stage of the biogas production process. Most biogas plants use a mixture of silage and manure as substrate at the initial stage of the biogas production process. A volume ratio of 80:20 to 50:50 of silage:manure is commonly used. Once the biogas production process has been started and is running, 100% fresh plant material and/or silage can be fed to the digester. More than 75% of the silage used is maize and less than 25% is grass. Among the different types of manures, the most biogas production is obtained from pig manure because monogastrics are less efficient at digestion.

Biogas generation is one of the most efficient technologies for generating renewable energy. It gives more energy per hectare than ethanol production. The process of biogas production utilizes the carbon from biomass and retains other nutrients which are returned to the farmland as fertilizer. Production of gas and energy from the biogas process is constant, unlike from solar power, wind energy or tidal power. Biogas production also provides an alternative earning opportunity for farmers.

In order to make biogas production more attractive economically to gain wider practice, there is a need in the art to improve the biogas production process, including increasing the efficiency of biogas production and reducing production cost.

The article (Lehtomaki et al: Laboratory investigations on co-digestion of energy crops residues with cow manure for methane production: effect of crop to manure ratio, Resources conservation and recycling, vol. 51, Jun. 19, 2007, pages 591-609) describes that co-digestion with cow manure could increase methane production (see abstract). The lactic acid bacterium Lactobacillus rhamsonus is used for the production of the silage.

It is here relevant to note that the Lehtomaki et al article does NOT describe anything about if use of Lactobacillus rhamsonus could give increased methane production—the article ONLY analyzes the effect of addition of the cow manure.

The article (Hassanat et al.: Effects of inoculation on ensiling characteristics, chemical composition and aerobic stability of regular and brown midrib silages, Animal feed science and technology, vol. 139, Nov. 16, 2007, pages 125-140) relates to production of the silage as such—i.e. it does NOT relate to a process for producing biogas as such.

The Hassanat et al article used Lactobacillus plantarum for the production of the silage. It is here relevant to note that the conclusion of the article (p138) concludes that the addition/use of Lactobacillus plantarum did NOT give relevant significant positive results—quite to the contrary addition/use of Lactobacillus plantarum reduced aerobic stability of the silage.

Similar to the Hassanat et al article—the article (Weinberg et al.: The effect of applying lactic acid bacteria at ensiling on the aerobic stability of silages, Journal of applied bacteriology, vol. 75, Jan. 1, 1993, pages 512-518) also relates to production of the silage as such—i.e. it does NOT relate to a process for producing biogas as such.

The printed copy of the article (Pakarinen et al: Storing energy crops for methane production: Effects of solids content and biological additive, Bioresource technology, vol. 99, Oct. 1, 2008, pages 7074-7082) was published 1 Oct. 2008. However, on the front page of the article is said “Available online 6 Mar. 2008”. Presently, it is not known if the 6 Mar. 2008 online version were identical to the later 1 Oct. 2008 published printed copy of the article.

The printed copy of the Pakarinen et al article relates to storing energy crops for methane production. The plant used was a mixture of grasses (see abstract). Lactobacillus plantarum was added/used as a so-called “biological additive” (see point “2.2 Laboratory trials” on page 7075-7076). The conclusion of the article was that biological additive (e.g. Lactobacillus plantarum did NOT assist in preserving the methane CH₄ yields (see last line of abstract).

### SUMMARY OF THE INVENTION

The problem to be solved by the present invention is to provide an IMPROVED process for producing biogas—i.e. giving increasing methane (CH₄) biogas yields.

The solution may be seen as based on that the present inventors have identified that addition/use of Lactobacillus plantarum lactic acid bacterium may give a significant increase in methane (CH₄) biogas yields, for instance when the plant is maize—see e.g. working examples herein for further details.

Accordingly, a first aspect of the invention relates to a process for producing biogas,
characterized by the presence of a Lactobacillus plantarum lactic acid bacterium, wherein the process comprises the steps of:
(a) producing a silage;
(b) mixing the silage of (a) with a starter in an anaerobic digester;
(c) contacting the silage of (a) with a Lactobacillus plantarum lactic acid bacterium, prior to, simultaneously with, or after step (b); and
(d) collecting biogas generated in the anaerobic digester.

An embodiment of the invention relates to the process of first aspect, comprising the steps of:
(i) producing the silage of step (a) of first aspect according to a process comprising the steps of:
   (1) providing a chopped plant and/or plant part;
   (2) contacting the chopped plant and/or plant part of (a) with a Lactobacillus plantarum lactic acid bacterium; and
   (3) storing the mixture of (b) in an air-tight silo;
   wherein the plant or plant part is suitable for silage production.

In certain embodiments, the process for producing biogas further comprises the step of mixing the silage of (a) with a biomass prior to, simultaneous with, or after step (b), wherein the biomass is selected from plant and/or plant part, dung, manure, industrial waste, or municipal waste, preferably from manure, industrial waste, or municipal waste.

In the process for producing a silage or biogas, the lactic acid bacteria is selected from Lactobacillus, Leuconostoc, Pediococcus, Lactococcus, Streptococcus, Propionibacterium, or any combinations thereof; preferably from Lactobacillus, Lactococcus, Enterococcus, Pediococcus, or any combinations thereof; more preferably from Lactobacillus plantarum, Lactobacillus brevis, Lactobacillus pentosus, Lactobacillus buchneri, Lactococcus lactis, Enterococcus faecium, Pediococcus pentasaceus, or any combinations thereof; even more preferably from Lactobacillus plantarum; most preferably from Lactobacillus plantarum CHCC6072 with the accession No. DSM16568, a variant thereof having similar characteristics, or any combinations thereof.

In the process for producing a silage or biogas, the Bacillus is selected from Bacillus licheniformis, Bacillus subtilis, Bacillus megaterium, Bacillus coagulans, Bacillus pumilus, Bacillus cereus var. toyoi, or any combinations thereof.

In the process for producing a silage or biogas, the plant is selected from the group consisting of grass, clover, maize, corn, lucerne, alfalfa, rye, barley, oats, wheat, triticale, beans, sorghum, sun flower, radish, artichoke, peas, sugar beets, and any combinations thereof; the plant part is selected from stalk, leave, kernels, or any combinations thereof.

In the process for producing a silage or biogas, 1 gram of the chopped plant and/or plant part is contacted with 10,000-1000,000 CFU, preferably 20,000-4000,000 CFU, more preferably 30,000-200,000, even more preferably 40,000-100,000 CFU, most preferably 50,000 CFU of the lactic acid bacteria or Bacillus.

The present invention provides the use of a lactic acid bacterium and optionally a Bacillus for producing a silage, wherein the silage is for use in biogas production.

The present invention also provides the use of a lactic acid bacterium and optionally a Bacillus for producing biogas.

The present invention provides a silo comprising a chopped plant or plant part and a lactic acid bacterium and optionally a Bacillus, wherein the plant or plant part is suitable for silage production.

The present invention further provides a silage comprising a chopped plant or plant part and a lactic acid bacterium and optionally a Bacillus, wherein the plant or plant part is suitable for silage production.

### BRIEF DESCRIPTION OF THE FIGURES

FIG. 1. A diagram of the biogas production process.

FIG. 2. 1.5 liter capacity glass silos compression gadget.

FIG. 3. Storage of silos at 25° C.

FIG. 4. Batch fermentation test facility according to VDI 4630.

FIG. 5. ODMl_{C} biogas yield from different inoculants (A-D) and control in fermentation test-batch procedure.

FIG. 6. ODMl_{C} methane yield from different inoculants (A-D) and control in fermentation test-batch procedure.

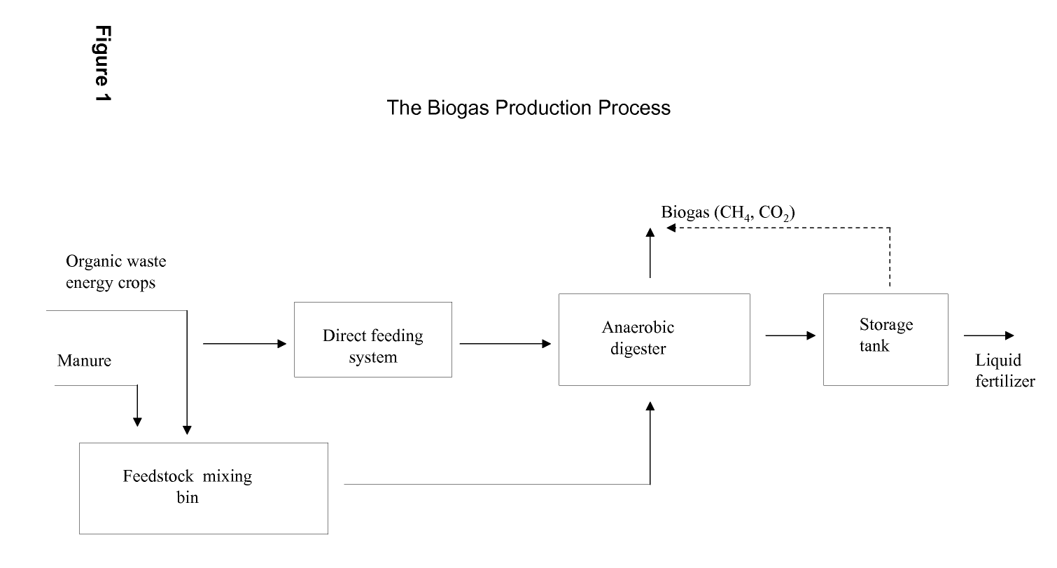

### DETAILED DESCRIPTION OF THE INVENTION

The terms used herein shall have the same meanings as those established in the art unless otherwise noted. The techniques mentioned herein shall be standard in the art unless otherwise noted.

The articles “a” and “an” are used herein to refer to one or to more than one (i.e., at least one) of the grammatical object of the article unless otherwise noted. By way of example, “an element” means one element or more than one element.

### Process for Producing a Silage

The invention provides a process for producing a silage, which is characterized by the presence of a lactic acid bacterium and optionally a Bacillus.

Specifically, the process comprises the steps of:
(a) providing a chopped plant and/or plant part;
(b) contacting the chopped plant and/or plant part of (a) with a lactic acid bacterium and optionally a Bacillus; and
(c) storing the mixture of (b) in an air-tight silo;
   wherein the plant or plant part is suitable for silage production.

Plants and plant parts which are suitable for silage production are known in the art. “Plant” is used herein in a general sense to refer to all parts of a plant including kernels. Suitable plants include, but are not limited to, grass, maize, corn, Lucerne, alfalfa, clover, beans, wheat, rye, barley, oats, triticale, sorghum, sun flower, peas, radish, sugar beets, and artichoke. Suitable plant parts include, but are not limited to, leave, stalk, kernels, and parts that are left over after harvest of the kernel or grain.

Plants and/or plant parts having high organic dry matter (ODM) content and low cellulose and lignin fractions are preferred. The ODM, cellulose and lignin contents of a plant or plant part can be readily determined by a skilled person using standard techniques, for example, those described in Herrmann C et al. (2007) and in the Examples below.

Preferred plants include, but are not limited to, maize, sorghum, triticale, barley, wheat, and grass. The most preferred plants are maize and grass.

One type of plant or plant part may be used alone. More than one type of plant and/or plant part may be used in combination. Preferred combinations include grass/clover, peas/beans cereals/peas/beans, and alfalfa/grass. The preferred volume or weight ratios of the different plants used in the combinations are known in the art and can be empirically determined by a skilled person.

The plant and/or plant part may be freshly harvested or previously harvested and wilted or partially dried. Preferably, the plant and/or plant part has a moisture content of about 40% to 75%. The preferred moisture content varies with the plant and/or plant part used. For example, the preferred moisture content is 60-75% for grass, 40-70% for Alfalfa, 65-72% for maize, and 45-65% for Lucerne.

The plant and/or plant part can be chopped by any suitable means known in the art to any size known in the art to be suitable for silage production.

As used herein, the expression “a lactic acid bacterium and optionally a Bacillus” means “a lactic acid bacterium, or a lactic acid bacterium and a Bacillus”.

Lactic acid bacterium is comprised in the order Lactobacillales which includes genera Lactobacillus, Leuconostoc, Pediococcus, Lactococcus, Streptococcus, Aerococcus, Carnobacterium, Enterococcus, Oenococcus, Teragenococcus, Vagococcus, Propionibacterium and Weisella.

The lactic acid bacterium is preferably selected from Lactobacillus, Leuconostoc, Pediococcus, Lactococcus, Streptococcus, Enterococcus, Propionibacterium, or any combinations thereof. More preferably, the lactic acid bacterium is selected from Lactobacillus, Lactococcus, Enterococcus, Pediococcus, or any combinations thereof. More preferably, the lactic acid bacterium is selected from Lactobacillus plantarum, Lactobacillus brevis, Lactobacillus pentosus, Lactobacillus buchneri, Lactococcus lactis, Enterococcus faecium, Pediococcus pentasaceus, or any combinations thereof. Even more preferably, the lactic acid bacterium is Lactobacillus plantarum. Most preferably, the lactic acid bacterium is selected from Lactobacillus plantarum CHCC6072 (item number: 689603, Chr. Hansen, Hørsholm Denmark; accession No. DSM16568 at the Deutsche Sammlung von Mikroorganismen and Zellkulturen (DSMZ)), a variant thereof having similar characteristics, or any combinations thereof. In another preferred embodiment, the lactic acid bacterium is a Lactococcus plantarum having similar characteristics as CHCC6072,

As established in the art and used herein, “a variant” refers to a microorganism, in particular, a strain, which is derived from a parent microorganism, in particular, a parent strain. A variant may be obtained by letting the parent microorganism undergo spontaneous mutation or subjecting the parent microorganism to mutagenesis treatments.

Mutagenesis treatments include, but are not limited to random mutagenesis and site-directed mutagenesis. Random mutagenesis can be carried out using mutagens including, but not limited to, chemical mutagens and UV light. Examples of chemical mutagens include 2-amino purine, ICR-191, nitrosoguanidine, hydroxylamine, and ethane methyl sulphonate (EMS).

Characteristics may include morphology, growth requirements and kinetics, and metabolic activity. These characteristics can be readily determined by a skilled person using established methods and assays in the art. The most important or determining characteristic is the capability of the microorganism to increase biogas production when the microorganism is used in silage production and/or the subsequent biogas production.

A microorganism is said to have “similar characteristics” as CHCC6072 if its capability of increasing biogas production is at least 50%, 60%, preferably at least 70%, 80%, more preferably at least 90%, 95% of that of CHCC6072. Most preferably, the microorganism is more effective at increasing biogas production than CHCC6072.

The capability of a microorganism to increase biogas production is defined as the percentage increase in biogas production in the presence of the microorganism vs. in its absence. The capability of a microorganism to increase biogas production can be determined by the methods described in Examples 1 and 2 or any equivalent established methods. In the event that different results are obtained by different methods, the results obtained by the methods described in Examples 1 and 2 will be used.

A strain of lactic acid bacterium may be used alone, or in combination with one or more strain(s) of lactic acid bacterium.

The Bacillus is preferably selected from Bacillus licheniformis, Bacillus subtilis, Bacillus coagulans, Bacillus pumilus, Bacillus cereus var. toyoi, or Bacillus megaterium.

A strain of Bacillus may be used alone, or in combination with one or more strain(s) of Bacillus.

Preferably, spores of the Bacillus are used.

One or more strain(s) of lactic acid bacterium may be used either with or without one or more strain(s) of Bacillus.

Preferably, 10,000-1,000,000 CFU, 20,000-400,000 CFU, more preferably 30,000-2,000,000 CFU, even more preferably 40,000-100,000 CFU, most preferably 50,000 CFU of a lactic acid bacterium are added to 1 gram (fresh weight) of chopped plant and/or plant part.

Preferably, 10,000-1,000,000 CFU, 20,000-400,000 CFU, more preferably 30,000-2,000,000 CFU, even more preferably 40,000-100,000 CFU, most preferably 50,000 CFU of a Bacillus are added to 1 gram (fresh weight) of chopped plant and/or plant part.

The colony forming unit of a lactic acid bacterium or a Bacillus preparation can be readily determined by a person skilled in the art using standard methods such as those described in the Examples.

When more than one strain of microorganism is used, the concentration (i.e., CFU/g chopped plant and/or plant part) described above applies to each strain.

The one or more strain of lactic acid bacterium and/or the one or more strain of Bacillus may be used at the same or different concentration(s) (i.e., CFU per gram (fresh weight) of a chopped plant and/or plant part).

Typically, the lactic acid bacterium is sprayed onto the plant and/or plant part during chopping. The weight of the chopped plant and/or plant part does not have to be exact. As used herein, “1 gram” means “approximately 1 gram” or “about 1 gram”, which in turn means 1±0.5 gram, preferably 1±0.25 gram, more preferably 1±0.1 gram. The weight of the chopped plant and/or plant part can be determined before, during or after chopping as the change in weight before and after chopping is less than 50% of the initial weight.

The lactic acid bacterium and/or the Bacillus used may be prepared in any form as long as it retains its ability to mediate the anaerobic digestion or fermentation of the plant and/or plant part. The lactic acid bacterium and/or the Bacillus may be in the form of a fresh live culture, rehydrated lyophilized bacterial preparation, or thawed frozen bacterial preparation. Preferably, the lactic acid bacterium and/or the Bacillus used are rehydrated lyophilized bacterium.

The methods for preparing a silo are known in the art. Examples include the methods disclosed in McDonald et al. (1991).

By “air-tight”, it is meant that the silo is sealed off so that there is very limited, if any, entry of air into the silo. The term “air-tight” is not an absolute term; it does not mean that there is absolutely no gas exchange between the inside and the outside of the silo. Methods for preparing an air-tight silo are known in the art. Examples include the methods disclosed in McDonald et al. (1991).

The ensiling process in the silo generally takes place at ambient temperature of 10-40° C. The temperature inside the silo may rise above this temperature during the ensiling process.

The silo may be stored for a period of few weeks up to 3 years at temperatures from −15° C. to 45° C.

The silage produced may be used for biogas production.

### Process for Producing Biogas

The invention provides a process for producing biogas, which is characterized by the presence of a lactic acid bacterium and optionally a Bacillus.

In one embodiment, the process comprises the steps of:
(a) producing a silage;
(b) mixing the silage of (a) with a starter in an anaerobic digester;
(c) contacting the silage of (a) with a lactic acid bacterium and optionally a Bacillus, prior to, simultaneously with, or after step (b); and
(d) collecting biogas generated in the anaerobic digester.

The process may further comprise the step of mixing the silage of (a) with a second biomass prior to, simultaneously with, or after step (b).

The silage may be produced in the presence or absence of an added lactic acid bacterium, or an added lactic acid bacterium and an added Bacillus. In a preferred embodiment, the silage is produced in the presence of an added lactic acid bacterium, or an added lactic acid bacterium and an added Bacillus.

In another embodiment, the process comprises the steps of:

(a) producing a silage according to the process described above;
(b) mixing the silage of (a) with a starter in an anaerobic digester; and
(c) collecting biogas generated in the anaerobic digester.

The process may further comprise the step of mixing the silage of (a) with a second biomass prior to, simultaneously with, or after step (b).

The process may further comprise the step of contacting the silage of (a) with a lactic acid bacterium and optionally a Bacillus, prior to, simultaneously with, or after step (b). In general, a strain of lactic acid bacterium may be used alone, or in combination with one or more strain(s) of lactic acid bacterium. A strain of Bacillus may be used alone, or in combination with one or more strain(s) of Bacillus. One or more strain(s) of lactic acid bacterium may be used with or without one or more strain(s) of Bacillus. The preferred lactic acid bacterium and/or Bacillus are as described previously.

One or more type(s) of silage may be used in the process. The more than one type of silage may differ in the plant or plant part used and/or the ensiling conditions which include, but are not limited to, method and condition used for filling the silo, size of the silo, presence or absence of added microorganism(s), the type and quantity of microorganism(s) added, storage length and conditions, type of machinery, plant buffer capacity, plant dry matter, compression or chopping length.

Each type of silage may contain one or more type(s) of plant and/or plant part. Preferred plant and/or plant part are as described above.

Silage may be used alone, or may be used in combination with one or more other type(s) of biomass (i.e., “a second biomass”). The more than one other type of biomass may be plant or plant part, dung, manure, industrial waste (e.g., stillage, pomace, whey, vegetable residues, grease trap contents), and municipal waste. The plant and/or plant part may be fresh, partially wilted, previously processed or left behind as waste in various industries, such as the food and the feed industry. Examples of waste include plant material waste and vegetarian food waste.

Preferably, silage is used in combination with manure which is suitable for biogas production. A person skilled in the art knows which manures are suitable for biogas production. Preferred manure includes pig manure and cattle manure.

The volume ratio of silage:manure is preferably in the range of 100:0 to 5:95, more preferably in the range of 80:20 to 50:50.

Preferably, the silage and the one or more other type(s) of biomass have 40-95% moisture content. The moisture content of a biomass can be readily determined by a skilled person using standard methods in the art.

A starter, also called an inoculum, can be any substance which is capable of mediating the anaerobic digestion or fermentation of the silage. The starter may be manure, including, but not limited to pig manure and cattle manure. The starter may be a sludge after previous anaerobic digestion, such as that obtained from an anaerobic digester during or after biogas production.

The volume ratio of starter to total biomass (i.e., substrate) is preferably in the range of 85:15 to 99:1, more preferably in the range of 90:10 to 98:2, even more preferably in the range of 95:5 and 97:3. Typically, the starter cattle manure is mixed with fresh organic material (i.e., biomass) at a volume ratio of 96:4.

The construction and the use of anaerobic digesters are known to those skilled in the art.

The methods and conditions for anaerobic digestion or fermentation are known in the art.

Typically, the anaerobic digester has to be “fed” every day with new biomass. New biomass or “feed” is added at least 4 times/day, preferably every 2 hours at a rate of 2% (volume) new biomass/day. The new biomass may be the same as or different from the biomass that is used to initiate the biogas production process. For example, a mixture of silage and manure may be used as the initial substrate at the beginning of a biogas production process. Once the process has been started and is running, silage and/or fresh plant or plant part may be used as the feed.

Fermentation in the digester is typically carried out at a mesophilic temperature of 38-40° C.

The biogas generated can be collected in any suitable way known in the art.

The biogas produced may be fed directly into the gas grid or be used to generate electricity.

### Use

The invention provides the use of a lactic acid bacterium and optionally a Bacillus for producing a silage and biogas.

A strain of lactic acid bacterium may be used alone, or in combination with one or more strain(s) of lactic acid bacterium. A strain of Bacillus may be used alone, or in combination with one or more strain(s) of Bacillus. One or more strain(s) of lactic acid bacterium may be used with or without one or more strain(s) of Bacillus. The preferred lactic acid bacterium and/or Bacillus are as described previously.

### Silo and Silage

The invention provides a silo comprising a chopped plant or plant part and a lactic acid bacterium and optionally a Bacillus, wherein the plant or plant part is suitable for silage production.

As established in the art and used herein, the term “silo” refers to a structure containing a plant and/or a plant part in which the ensilage process takes place and a silage is produced. In general, a silo comprises an outer wall with the plant and/or plant part enclosed in it. The plant and/or plant part are usually chopped. The outer wall of a silo may be solid or flexible. It may be built from plastic sheets, plastic stretch film wraps, concrete, steel or wood. Commonly used commercial silos include, but are not limited to, stack or clamp silo with or without retaining walls, tower silo, surface-walled clamp or bunker silo, flexible-walled silo, vacuum silo, plastic sausage silo, trench and big bale.

A silo may contain one type of plant or plant part, or a mixture of more than one type of plant and/or plant part. The plant or plant part in a silo may be newly filled into the silo, compressed and airtight covered and has not undergone any anaerobic digestion; it may be at any stage of the ensiling process and is partially digested to varying degrees; or it may be at the end of the ensiling process.

The invention also provides a silage comprising a chopped plant or plant part and a lactic acid bacterium and optionally a Bacillus, wherein the plant or plant part is suitable for silage production.

As established in the art and used herein, the term “silage” refers to the material produced by the ensilage or ensiling process which is the controlled fermentation of a plant and/or plant part of high moisture content. The plant and/or plant part comprised in a silage is fermented, i.e., has undergone anaerobic fermentation.

The silo and/or silage may contain one or more strain(s) of lactic acid bacterium with or without one or more strain(s) of Bacillus.

The silo and/or silage may contain one or more type(s) of plant and/or plant part.

The preferred plant or plant part, lactic acid bacterium, Bacillus, and ratio of lactic acid bacterium and/or Bacillus to plant or plant part are as described above.

The present invention is further illustrated by the following examples.

### EXAMPLES

### Example 1

### Ensiling of Maize

### 1.1 Materials and Methods

### 1.1.1 Ensiling

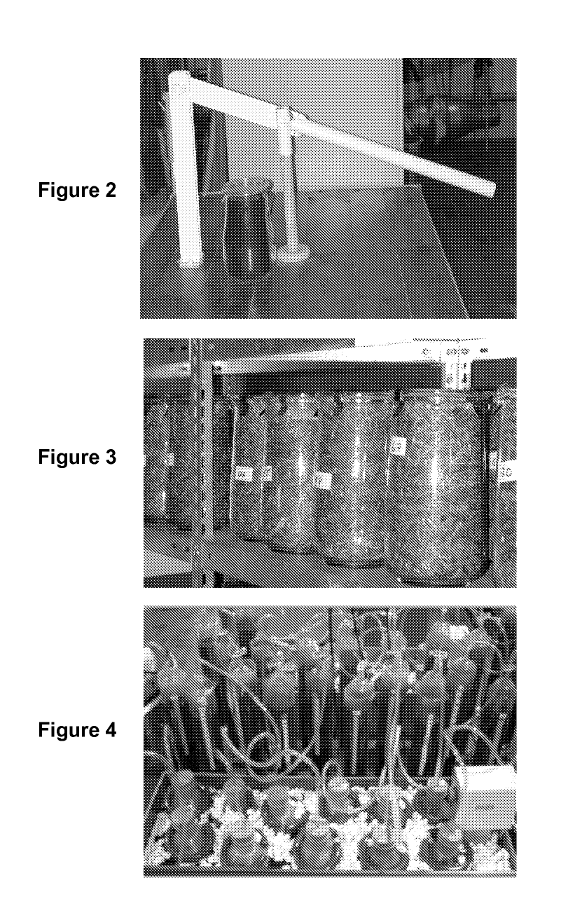

The maize was harvested on 1 Oct. 2007 in the “agt Agrargenossenschaft Trebbin”, Brandenburg. In order to investigate the impact of ensiling on the biogas yield, on 2^{nd} October freshly harvested and chopped material was pressed in 1.5-liter laboratory scale silos (Weck, Wehr-Oftlingen, Germany, FIGS. 2 & 23). Compression was done manually using a special pressing devise that ensured same conditions for all samples. The lab scale silos were stored at 25° C. for a defined period of 49 days. For each treatment, silage preparation was performed in 4 replicates. All silos were weighed after filling and before opening in order to determine conservation losses during ensiling process as the difference between the weights.

The trial was carried out in such a way that it conforms to DLG (Deutsche Landwirtschafts-Gesellschaft e.V.) Guidelines for silage trials and in line with the DLG silage additive approval scheme (DLG, 2006).

The following treatments were applied to fresh forages:
sample “Control”: without additives
sample “A”: Biomax® HMC (Inoculant A) @ 1 gram per ton forage (fresh weight) to apply 50.000 CFU of Enterococcus faecium DSM 16573+50,000 CFU of Lactobacillus plantarum DSM 16682/g of maize.
sample “B”: Inoculant B @ 1 gram per ton forage (fresh weight) to apply 50,000 CFU of Bacillus licheniformis DSM 5749+50,000 CFU of Lactobacillus plantarum DSM 16568 (CHCC6072)/g of maize.
sample “C”: Inoculant C @ 1 gram per ton forage (fresh weight) to apply 50,000 CFU of Lactobacillus brevis DSM 16570+50.000 CFU of Lactobacillus plantarum DSM 16568 (CHCC6072)/g of maize.
sample “D”: Inoculant D @ 1 gram per ton forage (fresh weight) to apply 50,000 CFU of Lactobacillus plantarum DSM 16568 (CHCC6072)/g of maize.

The inoculants are individually prepared for application, by suspending 1 gram/ton in 2 liters of distilled water and then evenly applying 2 ml of solution/kg of forage (fresh weight). 0.2 ml of distilled water should be applied/kg to the untreated maize.

### 1.1.2 Analytical Methods

Samples of silages were stored at −18° C. directly after taking material from lab scale silos for intended analysis and batch anaerobic digestion tests.

The contents of organic acids and alcohols except lactic acid were analyzed by gas chromatography (FISIONS) using a DB-FFAP fused silica capillary column (J&W scientific, 30 m×0.53 mm), helium as a carrier gas and a flame ionization detector for detection. For determination of lactic acid and sugar a high performance liquid chromatograph (DIONEX) equipped with a Eurokat H column (KNAUR, 300×8 mm) was used. It operated with 0.01N H₂SO₄ as a solvent at a flow rate of 0.8 ml/min. Detection was conducted by a refractive index detector RI 71 (SHODEX). Alcohol content was calculated as sum of ethanol and propanol, acetic acid is presented as sum of acetic and propionic acid in this study. Butyric acid, isobutyric acid, valerian acid, isovaleric acid and caproic acid are summed up as butyric acid.

pH was measured with the measuring electrode Sen Tix 41 (WTW).

Dry matter content of fresh material and silages was investigated by drying the material at 105° C. until the weight of the sample did not change any more. Since silages contain a greater amount of components that volatilize during drying process, DM was corrected as described by Weissbach et al. (1995).

Organic dry matter (ODM) was measured by determination of the ash content of dry samples in a muffle furnace at 550° C.

For further analysis defrosted plant material was dried at a temperature of 60° C. and grinded in a cutting mill (RETSCH). The content of total nitrogen (Nₜₒₜ) was determined using an elementar analyser (vario EL, Analysensysteme GmbH) operating at the principle of catalytical combustion under supply of oxygen and high temperatures. Elementar analysing was conducted according to the DUMAS method (DIN, 2006). Crude protein content was calculated as 6.25 multiplied by Nₜₒₜ.

Starch content was quantified according to the method of EWERS as described by Lengerken and Zimmermann. Measurement of starch content was done by detecting the optical rotation of a specially treated and filtrated dilution of the sample with a polarimeter (WOLFGANG GLOCK KG).

Lactobacillus was detected on Rogosa Agar (BD 248020; Merck 5413; Oxoid CM627) containing 0.4 g/l Actidione or 0.01% Delvocid (Delvocid Instant DSM 3-143-69-2/1). Specifically, viable counts of the silage samples are conducted as triplicates, where 10 grams of each sample are weighed into sterile stomacher bags and the sufficient amount of sterile diluent is added to make a 10× dilution. The sample is homogenized in a stomacher for 2 (possibly 4) minutes at 230 rpm. After preparing a dilution series, the inoculation is conducted by pour plating (3×1.0 ml of suitable dilution) or spread plating depending on the which inoculant the treatments contain. The plates are incubated aerobically at 30° C. for 72 hours. The analytical period from weighing out the sample until the samples are pour plated should not exceed 30 minutes.

For analyzing the treatments containing Enterococcus the following medium is used:

BEAA: Bile Esculin Azide Agar (e.g. Enterococcose™ Agar from Becton Dickinson and Company, Cockeysville, Md. 21030, USA; D-Coocose™ agar from bioMérieux sa, 69280 Marcy I'Etoile, France or from any other supplier who produces a medium of same composition:

| | | |
|---|---|---|
| Peptone 1 (pancreatic digest of casein) | 17.0 g/L | |
| Peptone 2 (peptic digest of meat) | 3.0 g/L | |
| Yeast extract | 5.0 g/L | |
| Ox bile (dehydrated) | 10.0 g/L | |
| Sodium chloride | 5.0 g/L | |
| Esculin | 1.0 g/L | |
| Ferric ammonium citrate | 0.5 g/L | |
| Sodium azide | 0.25 g/L | |
| Agar | 13.5 g/L | final pH 7.1 +/− 0.2 |

Inoculation is conducted by spread plating (3×0.1 mL from suitable dilutions) and the plates are incubated aerobically at 37° C. for 24 hours.

### 1.2 Results

### 1.2.1 Characterization of Fresh Material

Samples of fresh material were investigated for dry matter (DM), organic dry matter (ODM), pH, sugar, starch, lactic acid bacteria and buffer capacity.

TABLE 1
Chemical and microbiological composition of
fresh maize forages before ensiling, n = 2

| | | | | crude | | | | |
|---|---|---|---|---|---|---|---|---|
| | DM | ODM | pH | protein | sugar | starch | LAB | FC¹⁾ |
| Trial | % FM | % DM | — | % DM | % DM | % DM | cfu/g FM | — |
| Control | 36.70 | 95.08 | 4.27 | 8.88 | 7.83 | 24.12 | 5.1E+08 | 51 |
| A | 36.27 | 94.20 | 4.29 | 9.40 | 7.75 | 22.62 | 1.3E+09 | 47 |
| B | 38.22 | 93.23 | 4.30 | 8.39 | 6.84 | 25.81 | 4.8E+09 | 52 |
| C | 37.45 | 95.42 | 4.31 | 8.07 | 6.49 | 29.60 | 8.8E+08 | 60 |
| D | 37.70 | 95.27 | 4.32 | 8.17 | 7.35 | 27.09 | 6.4E+08 | 58 |

¹⁾Fermentabillity coefficient = DM + 8 (sugar/buffer capacity), (DLG, 2000)

The data from protein, sugar and starch of the fresh maize corresponded with data in the literature. The content of lactic acid bacteria in the control samples was very high. It may be that the storage conditions between harvest and trial was not optimal, so that the bacteria could increase in the fresh material. The same affects to the dry matter and the pH.

In the literature, the fermentabillity coefficient on fresh maize averages 58. If the coefficient is greater than 45, then the fermentation will be stable.

### 1.2.2 Characterization of Maize Silage

Samples of silages were investigated for dry matter (DM), organic dry matter (ODM), pH, sugar, lactic acid bacteria, organic acids and alcohols and also analyzed for ammonia-nitrogen (NH₄—N).

TABLE 2
Chemical and microbiological composition of
maize silages after 49 days ensiling, n = 4

| | | | | crude | | | |
|---|---|---|---|---|---|---|---|
| | DM_{c} | ODM_{c} | pH | protein | sugar | NH₄—N | LAB |
| Trial | % FM | % DM | — | % DM_{c} | % DM_{c} | % DM_{c} | cfu/g FM |
| Control | 37.02 | 95.88 | 3.82 | 8.38 | 2.86 | 0.08 | 2.15E+07 |
| A | 37.86 | 96.04 | 3.81 | 8.09 | 3.09 | 0.09 | 3.00E+07 |
| B | 37.77 | 95.99 | 3.84 | 8.30 | 2.22 | 0.08 | 1.30E+07 |
| C | 37.78 | 96.06 | 3.83 | 8.23 | 2.96 | 0.07 | 2.94E+07 |
| D | 37.60 | 96.00 | 3.85 | 8.42 | 2.54 | 0.07 | 3.75E+07 |

DM_{c} (corrected) = 2.22 + 0.96 × DM (WEISSBACH et al, 1995)

TABLE 3
Chemical composition, losses and evaluation
of maize silages after 49 days ensiling

| | Alc | LA | AA | BA | losses | Evaluation¹⁾ |
|---|---|---|---|---|---|---|
| Trial | % DM_{c} | % DM_{c} | % DM_{c} | % DM_{c} | % FM | Points/Mark |
| Control | 1.36 | 3.95 | 0.83 | 0 | 0.89 | 100/1 |
| A | 1.44 | 3.69 | 0.96 | 0 | 0.88 | 100/1 |
| B | 1.54 | 3.91 | 1.07 | 0 | 0.89 | 100/1 |
| C | 1.56 | 3.63 | 1.10 | 0 | 0.70 | 100/1 |
| D | 1.45 | 3.59 | 1.13 | 0 | 0.91 | 100/1 |

¹⁾Evaluation: DLG-Schlüssel (2006)

### Example 2

### Biogas Yield and Quality

### 2.1 Materials and Methods

Batch experiments were carried in four replicates with lab-scale vessels with a working volume of 2.0 liter according to the guideline VDI 4630 (2004). A constant temperature of 35° C. was maintained through a water bath (FIG. 4).

Sludge after anaerobic digestion of maize and animal slurry was used as inoculum (Table 4 for the current experiments. 1.5 kg of the stabilized inoculum was mixed with 0.05 kg maize silage for batch tests. The reactor vessels were connected with calibrated wet gas meters for measuring the biogas production up to 28 days. The biogas produced from ensiled material was measured daily during the digestion period and plotted as a cumulative curve related to corrected organic dry matter ODM_{C}. The content of methane in the biogas was analyzed by means of a gas analyzer (ANSYCO GA94) and results to cumulative methane yield. The volume of biogas was calculated to normalized liters (NI); (dry gas, t₀=273K, p₀=1013 hPa) and the methane and carbon dioxide content was corrected to 100% (headspace correction according to VDI 4630). The volume of biogas produced from the inoculum was subtracted from the batch tests with substrate.

TABLE 4
Chemical characterization of inoculum used for batch
anaerobic digestion tests

| Parameter | Unit | Mean |
|---|---|---|
| Dry matter | % FM | 2.65 |
| Organic dry matter | % DM | 49.24 |
| pH | — | 8.38 |
| NH₄—N | g/kg ww | 1.17 |
| Nₜₒₜ | g/kg ww | 2.05 |
| Organic acids | g/kg ww | 1.11 |

ww = wet weight

### 2.2 Results

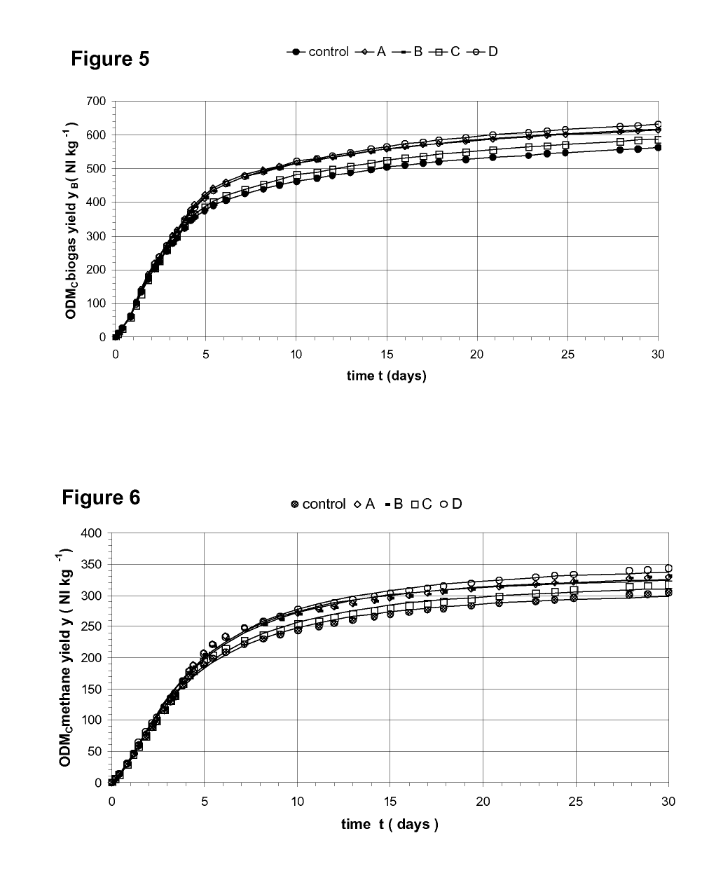

On basis of the cumulative curve of produced biogas (FIG. 5) and methane (FIG. 6), both the biogas- and methane yield can be detected according to VDI 4630.

The criterion for terminating the test is when the daily methane rate is equivalent to only 1% of the total volume of methane produced up to that time. In order to find out the exact methane yield an approximation of the cumulative curve is recommended. For this approximation a Hill-function type was used (1)

$$y = {y}_{max} \cdot \frac{{t}^{b}}{{c}^{b} + {t}^{b}} \qquad ( 1 )$$

where y is the methane yield at any time t, yₘₐₓ is the maximum methane yield for t→∞ and b and c are coefficients.

TABLE 5
Methane yield for 1% criterion according VDI 4630, maximum
methane yield for approximation according to Hill-equation and
related coefficients

| | y | t | | | |
|---|---|---|---|---|---|
| | for 1% criterion | for 1% criterion | yₘₐₓ | b | c |
| Trial | Ig⁻¹ | days | Ig⁻¹ | — | — |
| Control | 279 | 17 | 314 | 1.4234 | 3.9568 |
| A | 304 | 17 | 337 | 1.5756 | 3.9142 |
| B | 306 | 17 | 341 | 1.5119 | 4.0469 |
| C | 290 | 17 | 329 | 1.4273 | 4.1232 |
| D | 315 | 17 | 356 | 1.4595 | 4.2314 |

### 2.3 Conclusion

All silages were evaluated with the label/mark on. There was no significant difference in the quality between the control silage and the silages prepared with the addition of one or more microorganism. No butyric acid was detected in any of the samples, the content of acetic acid was below 3.0% DM and the pH was below 4.5 when the DM was in the range between 30% and 45%.

Surprisingly, silages prepared with the addition of one or more microorganism yielded more biogas and methane when used as a substrate in a biogas production process than the untreated control silage (FIGS. 5 & 6). In particular, sample D, which was prepared with the addition of Lactococcus plantarum DSM16568 alone, produced the highest methane yielded, which is 13% higher than that produced from the control silage.

### Example 3

### Economical Benefit

The typical methane yield is 100 m³/ton fresh matter (FM). 1 m³ methane produces 3.8 kwh electricity. In Germany a special price for electricity from biogas is ε0.16/kwh. A typical medium sized biogas plant uses about 10,000 tons of maize/year. A typical German household uses 3,600 kwh/year. Based on these facts and the results of Example 2, preparing silage from 10,000t maize with the addition of CHCC6072 would generate electricity for an extra 150 households!

TABLE 6
Return of investment using CHCC6072 in biogas production.

| | Untreated | CHCC6072 |
|---|---|---|
| m³ methane/kg FM | 98.25 | 112.5 (P < 0.001) |
| Electricity (Kwh) | 373.4 | 427.5 |
| Value of electricity ( [symbol] ) | 59.74 | 68.40 |
| Cost of additive ( [symbol] ) | 0.00 | 1.10 |
| Extra benefit/t ( [symbol] ) | | 7.56 |
| Extra benefit/10,000t ( [symbol] ) | | 75,600.00 |
| Return on investment | | 6.9:1 |

### REFERENCES

Deutsches Institut für Normierung e.V., (Norm-Entwurf) DIN EN ISO 16634, Cereals, pulses, milled cereal products, oilseeds and animal feeding stuffs—Determination of the total nitrogen content by combustion according to the Dumas principle and calculation of the crude protein content, Standard (2006).
DLG (editor), DLG-Richtlinien für die Prüfung von Siliermitteln auf DLG-Gütezeichen-Fähigkeit, 1.4.2000
DLG (editor), Grobfutterbewertung Teil B: DLG-Schlüssel zur Beurteilung der Gärqualität von Grünfuttersilagen auf der Basis der chemischen Untersuchung, DLG e.V.—Ausschuss für Futterkonservierung, DLG-Information (February 2006).
Herrmann C et al. (2007) Parameters influencing substrate quality and biogas yield. Proceedings, 15th European Biomass Conference & Exhibition “From Research to Market Deployment”, 7-11 May 2007, Berlin, Germany, Maniatis, K. et al. eds., page 809-819
Lengerken J v & Zimmermann K (1991) Handbuch Futtermittelprüfung, Deutscher Landwirtschaftsverlag Berlin GmbH
McDonald P et al. (1991) The Biochemistry of Silage, 2nd Ed., Chr. Hansen Biblioteket.
VDI-Gesellschaft Energietechnik/Fachausschuss Regenerative Energien, VDI 4630, Fermentation of organic materials, Characterisation of the substrate, sampling, collection of material data, fermentation tests, Verein Deutscher lngenieure, Düsseldorf (2006).
Weissbach F. Kuhla S. (1995). Substance losses in determining the dry matter content of silage and green fodder: arising errors and possibilities of correction. Übersicht Tieremährung 23, pp. 189-214.

1. A process for producing biogas, characterized by the presence of a Lactobacillus plantarum lactic acid bacterium, wherein the process comprises the steps of:
(a) producing a silage;
(b) mixing the silage of (a) with a starter in an anaerobic digester;
(c) contacting the silage of (a) with a Lactobacillus plantarum lactic acid bacterium, prior to, simultaneously with, or after step (b); and
(d) collecting biogas generated in the anaerobic digester.

2. The process of claim 1, comprising the steps of:
(i) producing the silage of step (a) of claim 1 according to a process comprising the steps of:
(1) providing a chopped plant and/or plant part;
(2) contacting the chopped plant and/or plant part of (a) with a Lactobacillus plantarum lactic acid bacterium; and
(3) storing the mixture of (b) in an air-tight silo;
wherein the plant or plant part is suitable for silage production.

3. The process of claim 1, further comprising the step of mixing the silage of (a) with a biomass prior to, simultaneous with, or after step (b), wherein the biomass is selected from plant and/or plant part, dung, manure, industrial waste, or municipal waste, preferably from manure, industrial waste, or municipal waste.

4. The process of claim 1, wherein the Lactobacillus plantarum lactic acid bacterium is Lactobacillus plantarum CHCC6072 with the accession No. DSM16568, or a variant thereof having similar characteristics.

5. The process of claim 4, wherein the Lactobacillus plantarum lactic acid bacterium is Lactobacillus plantarum CHCC6072 with the accession No. DSM16568.

6. The process of any claim 1, wherein there in step (c) of claim 1 is also contacting the silage with a Bacillus.

7. The process of claim 6, wherein the Bacillus is selected from Bacillus licheniformis, Bacillus subtilis, Bacillus megaterium, Bacillus coagulans, Bacillus pumilus, Bacillus cereus var. toyoi, or any combinations thereof.

8. The process of any claim 1, wherein the plant is selected from the group consisting of grass, clover, maize, corn, lucerne, alfalfa, rye, barley, oats, wheat, triticale, beans, sorghum, sun flower, radish, artichoke, peas, sugar beets, or any combinations thereof.

9. The process of claim 8, wherein the plant is maize.

10. The process of claim 1, wherein the plant part is selected from stalk, leave, kernels, or any combinations thereof.

11. The process of claim 1, wherein 1 gram of the chopped plant and/or plant part is contacted with 10,000-1,000,000 CFU, preferably 20,000-4,000,000 CFU, more preferably 30,000-200,000, even more preferably 40,000-100,000 CFU, most preferably 50,000 CFU of the lactic acid bacteria.
